(19) United States
(12) Patent Application Publication
(10) Pub. No.: US 2026/0001858 A1
(43) Pub. Date: Jan. 1, 2026

(54) DEVICE SYSTEM AND METHOD FOR PREPARING CYCLIC CARBONATE BY DIOL ESTERIFYING CYCLIZATION

(71) Applicants: HUIZHOU INSTITUTE OF GREEN ENERGY AND ADVANCED MATERIALS, Huizhou, Guangdong (CN); INSTITUTE OF PROCESS ENGINEERING, CHINESE ACADEMY OF SCIENCES, Beijing (CN)

(72) Inventors: Songsong CHEN, Huizhou, Guangdong (CN); Junping ZHANG, Huizhou, Guangdong (CN); Hanwei HU, Huizhou, Guangdong (CN); Li DONG, Huizhou, Guangdong (CN); Huanhuan ZHAO, Huizhou, Guangdong (CN); Suojiang ZHANG, Huizhou, Guangdong (CN)

(21) Appl. No.: 18/879,729

(22) PCT Filed: Dec. 11, 2023
(86) PCT No.: PCT/CN2023/137793

§ 371 Date: Dec. 27, 2024

(30) Foreign Application Priority Data
Nov. 21, 2023 (CN) 202311563550.X

(51) Int. Cl. C07D 317/38; B01J 35/50

(52) U.S. Cl. CPC C07D 317/38; B01J 35/50

(57) ABSTRACT

Provided are a device system and a method for preparing cyclic carbonate by diol esterifying cyclization. The device system comprises a reaction unit, a gas-liquid separation unit, and a refining unit which are connected in sequence; in the reaction unit, a nitrile compound is used as an auxiliary to synthesize cyclic carbonate by diol esterifying cyclization with CO₂; the reaction unit comprises any one of a fixed-bed reactor, a bubbling-bed reactor, or a fluidized-bed reactor; and a feeding/discharging method of liquid of the reaction unit comprises top liquid-feeding and bottom liquid-discharging, or bottom liquid-feeding and top liquid-discharging; the gas-liquid separation unit comprises a first separation device and a second separation device which are arranged in series; the refining unit comprises a light-component removal tower, a heavy-component removal tower, and a high-purity tower which are arranged in sequence. In the present application, a nitrile compound is used as an auxiliary to synthesize cyclic carbonate by efficient diol esterifying cyclization, which has the advantages of safe production process, high conversion, and simple operation, and provides a transformative new route for industrial preparation of cyclic carbonate.

### TECHNICAL FIELD

The present application relates to the technical field of cyclic carbonate preparation, such as a device system and a method for preparing cyclic carbonate by diol esterifying cyclization.

### BACKGROUND

Cyclic carbonate is mainly five-membered cyclic carbonate, and has excellent properties such as high solubility, low toxicity, and stable chemical properties. Cyclic carbonate is widely used as chemical intermediate and aprotic polar solvents, specifically being applied to the electrolyte of lithium-ion batteries, and the organic synthesis field such as monomers of polycarbonate and polyurethane and intermediates of pharmaceuticals and other fine chemicals. It generally has the following general formula:

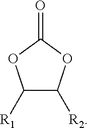

At present, there are three main methods for the industrial production of cyclic carbonate, including the phosgenation method, ester exchange method, cycloaddition method of CO₂ with epoxides, and urea alcoholysis method. The phosgenation method is the conventional synthesis method and is the earliest industrialized method for preparing cyclic carbonate, but it has been eliminated because of using high-toxic phosgene, causing serious environmental pollution, and failures in meeting green, environmental-friendly requirements. In the ester exchange method, the linear carbonate is used to react with polyol to synthesize cyclic carbonate under the action of catalysts. Because the reaction is reversible, there are problems such as a low yield of the product carbonate, etc. The method where CO₂ and epoxides are directly catalyzed to synthesize cyclic carbonate has a high atom utilization rate, and most existing cyclic carbonate preparation processes adopt this method, but the raw material epoxides have hazards such as flammability and explosiveness, the production process is dangerous, and the recovery and treatment of exhaust is difficult.

CN107915713A discloses a method for producing ethylene carbonate, and CN105541781A discloses a process for preparing cyclic carbonate. In both cases, ethylene oxide and CO₂ are utilized to generate cyclic ethylene carbonate under the action of catalysts, and the yield is high, but the production process has a great safety risk because ethylene oxide has explosion risk almost over the whole concentration range. CN115724819A discloses a device and a method for preparing ethylene carbonate. In this method, the low-concentration ethylene oxide and CO₂ are used to synthesize ethylene carbonate to reduce the safety risk of the production process, but a large amount of ethylene carbonate needs to be used as an absorption solution of ethylene oxide to provide the condition of low-concentration ethylene oxide, which has problems such as high investment in equipment and high operating load. CN104059047A discloses a continuous reaction process for synthesizing cyclic carbonate from urea. In this method, a tanks-in-series reaction device is used, and urea and polyol are used as raw materials to synthesize cyclic carbonate, and the yield reaches 96% or more, but the process is long, the operation is complicated, and the recovery of ammonia released during the reaction process increases the cost of energy consumption and the cost of environmental protection at the same time.

At present, the preparation of cyclic carbonate from diols at domestic and abroad is basically at the laboratorial stage, and has not been reported for its industrialization.

Huang Shiyong et al. have reported that CO₂ and 1.2-propylene glycol are reacted to prepare propylene carbonate under the conditions of anhydrous zinc acetate as a catalyst and acetonitrile as a solvent and a dehydrating agent, and the yield reaches 24.2%; the yield of the reaction of CO₂ and ethylene glycol is only 10.8% under the same reaction conditions (Synthesis of Cyclic Carbonate from Carbon Dioxide and Diols over Metal Acetates. S. Y. Huang et al. Journal of Fuel Chemistry and Technology, 35 (06) 701-70S). Du Ya et al. have reported that 1.2-propylene glycol and supercritical CO₂ are used to synthesize propylene carbonate catalyzed by organotin compounds, where the reaction is promoted by the addition of the co-solvent DMF and the dehydrating agent ketal to the reaction system: however, the reagents, propylene glycol and DMF, both require the pretreatments of dehydration and distillation before the reaction (Sn-catalyzed synthesis of propylene carbonate from propylene glycol and CO₂ under supercritical conditions. J. Mol. Catal. A-Chem., 241 (1-2) 233-237). Yu Na Lim et al. have reported that CO₂ and various alcohols are used to synthesize cyclic carbonate and linear carbonate without the addition of metal catalysts and inorganic bases, where the reaction system uses two equivalents of an organic base DBU as an auxiliary, an ionic liquid as a catalyst, and dibromomethane as a reaction solvent, and the yield of ethylene carbonate can reach 74% (Metal-Free Synthesis of Cyclic and Acyclic Carbonates from CO₂ and Alcohols. Yu Na Lim et al. Eur. J. Org. Chem . . . 2014 (9): 1823-1826). However, the reaction has problems such as the high price of dibromomethane and the recovery of ionic liquids. A. Brege et al. have reported that the coupling reaction of CO₂ and diols is promoted by using an organic dual system of a combination of organic base and organic halide, where two organic dual catalytic systems, DBU/EtBr and TEA/TsCl, are introduced, and when ethylene glycol is used as a reaction substrate, a selectivity of the ethylene carbonate is 69% and the yield is 44% (The coupling of CO₂ with diols promoted by organic dual systems: Towards products divergence via benchmarking of the performance metrics, A. Brege et al, J. CO₂ Util., 38:88-98); however, the two systems both produce a large number of by-products, and the by-products of the DBU/EtBr system are mainly bis-carbonate compounds.

In summary, at present, the preparation of cyclic carbonate by reacting epoxides with CO₂ is commonly used in industry, the flammable and explosive hazard of epoxy alkanes limit the market development of the technology, and the preparation route of cyclic carbonate by using CO₂ and diols is still in the stage of exploration of the basic conditions, and the process device and method have not yet been publicly disclosed. After years of unremitting research and exploration, our team has developed and proposed a safer, simpler, and transformative device and a method for preparing cyclic carbonate, wherein the high-purity cyclic carbonate is prepared by reacting CO₂ and diol as raw materials under the action of the nitrile compound as an auxiliary, thus realizing the transformative development of the carbonate industry.

### SUMMARY

The following is a brief summary of subject matter that is described in detail herein. This summary is not intended to be limiting as to the scope of the claims.

The present application provides a device system and a method for preparing cyclic carbonate by diol esterifying cyclization, which realizes the synthesis of cyclic carbonate by efficient diol esterifying cyclization with a nitrile compound as an auxiliary, and the method has a high conversion rate of diol, and has the advantages of safe process, simplicity and stable product quality, providing a transformative novel route for preparing cyclic carbonate.

In a first aspect, the present application provides a device system for preparing cyclic carbonate by diol esterifying cyclization: the device system comprises a reaction unit, a gas-liquid separation unit, and a refining unit which are connected in sequence:
in the reaction unit, a nitrile compound is used as an auxiliary to synthesize cyclic carbonate by diol esterifying cyclization with CO₂: the reaction unit comprises any one of a fixed-bed reactor, a bubbling-bed reactor, or a fluidized-bed reactor; and a feeding/discharging method of liquid of the reaction unit comprises top liquid-feeding and bottom liquid-discharging, or bottom liquid-feeding and top liquid-discharging:
the gas-liquid separation unit comprises a first separation device and a second separation device which are arranged in series:
the refining unit comprises a light-component removal tower, a heavy-component removal tower, and a high-purity tower which are arranged in sequence.

In the reaction unit of the device system for preparing cyclic carbonate by diol esterifying cyclization in the present application, the diol compound and CO₂ are used as reaction raw materials, and the nitrile compound is used as a dehydrating agent: the preparation of cyclic carbonate is safely and efficiently realized: the material from the outlet of the reaction unit is fed into the gas-liquid separation unit, which can not only improve the purity of cyclic carbonate, but also effectively reduce the loss of the raw material CO₂; and then the liquid phase is fed into the refining unit, so that the target product cyclic carbonate can further meet the requirement of high purity index. In the whole process, the conversion rate of the raw material diol is high, and the quality of cyclic carbonate is stable.

The light-component removal tower, the heavy-component removal tower, and the high-purity tower in the present application are all provided with a liquid inlet, a top liquid outlet, and a bottom liquid outlet, an internal part can be one or a combination of a filler or a column tray, and a bottom reboiler is any one of a kettle-type reboiler, a vertical thermosyphon reboiler, or a horizontal thermosyphon reboiler.

The light-component removal tower, the heavy-component removal tower, and the high-purity tower of the refining unit in the present application all operate in vacuum, and the tower tops are connected to vacuum pumps through pipelines, respectively. In order to reduce the material loss caused by vacuum pumping, a condenser, a gas-liquid separation device, and a mechanical pump are arranged on the pipeline connecting the top liquid outlet of the light-component removal tower and the liquid inlet of the reaction unit, which can remove some gas from the circulation of the nitrile compound and circulate the nitrile compound to the reaction unit for reuse: the pipeline after the mechanical pump is divided into two flows, one flow goes back to the top of the light-component removal tower via a pressure reducing valve, and the other flow circulates to the reaction unit to continue the reaction. A mechanical pump is arranged on the pipeline connecting the bottom liquid outlet of the light-component removal tower with the liquid inlet of the heavy-component removal tower.

In one embodiment, the reaction unit comprises any one of a jacketed heat exchanger, a shell-and-tube heat exchanger, or a built-in heat exchanger.

In one embodiment, when the reaction unit is a fixed-bed reactor, the feeding/discharging method is top liquid-feeding and bottom liquid-discharging.

In one embodiment, when the reaction unit is a bubbling-bed reactor, the feeding/discharging method is bottom liquid-feeding and top liquid-discharging.

In one embodiment, when the reaction unit is a fluidized-bed reactor, the feeding/discharging method is bottom liquid-feeding and top liquid-discharging.

In one embodiment, the reaction unit is provided with a heater for maintaining the temperature of the reaction process to ensure the reaction conversion efficiency, wherein a medium of the heater is any one of hot water, steam, or heat transfer oil.

In one embodiment, a gas outlet of the first separation device is connected to a gas inlet of the reaction unit via a compression device, so that the gas phase in the reaction material can be separated and circulated to the reaction unit for reuse.

In the present application, the pipeline connecting the gas outlet of the first separation device and the gas inlet of the reaction unit is also provided with a heat exchanger, which ensures that the temperature of the circulation gas before entering the compression device meets the requirements of safe operation.

In one embodiment, the pipeline connecting the liquid outlet of the second separation device and the liquid inlet of the light-component removal tower is also provided with a heat exchanger, which is used for controlling the thermal state of the material fed into the light-component removal tower.

In one embodiment, the liquid outlet of the light-component removal tower is connected to the liquid inlet of the reaction unit, so that the light component nitrile compound and diol compound which is not fully reacted, separated by the light-component removal tower, are circulated to the reaction unit, which can effectively improve the conversion rate of the raw material diol.

In one embodiment, the heavy-component removal tower is connected to a bottom part of the light-component removal tower.

Most of heavy components such as amide compounds separated by the heavy-component removal tower in the present application are fed into an auxiliary regeneration unit and regenerated as an effective auxiliary by catalytic dehydration. The heavy-component removal tower separates the product cyclic carbonate from a small amount of the heavy compound brought thereby: the high-purity cyclic carbonate is extracted from the side of the high-purity tower, the industrial-grade product is extracted from the tower top, and the heavy component at the tower bottom after analysis and detection can be circulated to the heavy-component removal tower to be reused.

In one embodiment, the pipeline connecting the bottom liquid outlet of the heavy-component removal tower and the bottom liquid inlet of the light-component removal tower is provided with a mechanical pump, and the liquid extracted from the bottom liquid outlet of the heavy-component removal tower is circulated to the bottom liquid inlet of the light-component removal tower after checked as qualified by the detection and analysis.

In one embodiment, the refining unit further comprises a crystallization device, which can avoid heavy-component byproducts, or light components that are difficult to separate due to the formation of azeotrope, entering the subsequent refining system and affecting the product quality.

In one embodiment, the crystallization device is arranged between the heavy-component removal tower and the high-purity tower.

In the present application, when a cyanopyridine solvent is used as an auxiliary for preparing cyclic propylene carbonate, since the cyanopyridine solvent is prone to form binary azeotrope with the cyclic propylene carbonate, it is necessary to provide a crystallization device between the heavy-component removal tower and the high-purity tower to realize the preparation of the high-purity cyclic propylene carbonate.

In one embodiment, the crystallization device comprises an evaporation crystallizer or a cooling crystallizer.

In one embodiment, the high-purity tower is connected to the heavy-component removal tower.

In one embodiment, the bottom liquid outlet of the heavy-component removal tower is connected to the auxiliary regeneration unit, which can realize the regeneration and recycling of the nitrile compound auxiliary. In the auxiliary regeneration unit, a high-efficient dehydrating agent can be used in cyanation of the amide compounds for regeneration and recycling, such as potassium oxide, sodium oxide, calcium oxide, phosphorus pentoxide and other strong dehydrating agents, and the amide compounds can be converted into a nitrile compound for further use.

In a second aspect, the present application also provides a method for preparing cyclic carbonate by diol esterifying cyclization, and the method is performed with the device system for preparing cyclic carbonate by diol esterifying cyclization according to the first aspect.

The method comprises using a nitrile compound as an auxiliary to realize the diol esterifying cyclization to synthesize cyclic carbonate, and the specific reaction is as follows:

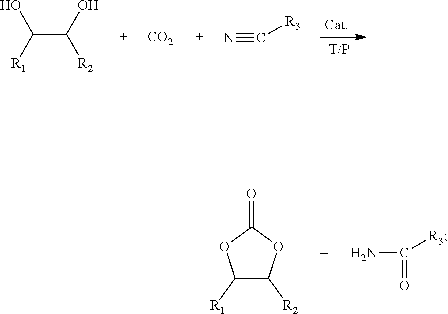

wherein the diol comprises a vicinal diol;
in the molecular structure of the diol, R₁ and R₂ comprise any one of hydrogen, methyl, ethyl, or propyl:
in the molecular structure of the nitrile compound, R₃ comprises any one of ethyl, phenylmethyl, pyridine, pyrimidine, pyrazine, imidazole, or quinoline.

In one embodiment, the method comprises the following steps:
(a) introducing CO₂ gas from a gas inlet of a reaction unit, and feeding a mixed solution of a diol and a nitrile compound from a liquid inlet of the reaction unit, and performing diol esterifying cyclization with CO₂ to form cyclic carbonate under the action of a catalyst in the reaction unit:
(b) feeding a reaction material extracted from the reaction unit in step (a) into a first separation device and a second separation device sequentially for gas-liquid separation, introducing a gas phase extracted from a gas outlet of the first separation device into a gas inlet of the reaction unit; and feeding a liquid phase extracted from a liquid outlet of the second separation device into a refining unit for refinement:
(c) subjecting a solution extracted from the liquid outlet of the second separation device in step (b) to a light-component removal tower, and extracting light components from a top liquid outlet of the light-component removal tower, and circulating to the reaction unit in step (a) for further reaction:
(d) feeding a cyclic carbonate solution extracted from a bottom liquid outlet of the light-component removal tower in step (c) into the heavy-component removal tower for removing heavy components, extracting a solution extracted from a top liquid outlet of the heavy-component removal tower or circulating to a bottom liquid inlet of the light-component removal tower for further separating light components out, and feeding a liquid phase extracted from the heavy-component removal tower into a high-purity tower for separation and purification and then extracting.

In one embodiment, the reaction unit is filled with a heterogeneous catalyst and/or a homogeneous catalyst.

In one embodiment, the heterogeneous catalyst comprises any one or a combination of at least two of silicon oxide, aluminum oxide, iron oxide, copper oxide, zinc oxide, tin oxide, lanthanum oxide, cerium oxide, cobalt oxide, dialkylzinc oxide, dialkyltin oxide, dialkyllanthanum oxide, dialkylcerium oxide, or dialkylcobalt oxide, wherein typical but non-limiting combinations comprise a combination of silicon oxide and aluminum oxide, a combination of iron oxide and copper oxide, a combination of zinc oxide and tin oxide, a combination of lanthanum oxide and cobalt oxide, or a combination of dialkylzinc oxide and dialkylcerium oxide.

In one embodiment, a shape of the heterogeneous catalyst comprises any one or a combination of at least two of powder, granule, sphere, rod, cube, or polyhedron, wherein typical but non-limiting combinations comprise a combination of powder and granule, a combination of sphere and rod, a combination of cube and powder, or a combination of polyhedron and sphere.

In one embodiment, the homogeneous catalyst comprises any one or a combination of at least two of zinc bromide, stannic bromide, cerium bromide, tetraalkyl phosphonium bromide, trialkylethyl phosphonium bromide, tetraphenyl phosphonium bromide, triphenylbutyl phosphonium bromide, zinc acetate, tin acetate, or cerium acetate, wherein typical but non-limiting combinations comprise a combination of zinc bromide and cerium bromide, a combination of tetraalkyl phosphonium bromide and trialkylethyl phosphonium bromide, a combination of tetraphenyl phosphonium bromide and zinc acetate, or a combination of tin acetate and tetraalkyl phosphonium bromide.

In one embodiment, the nitrile compound comprises any one or a combination of at least two of acetonitrile, cyanoquinoline, cyanopyridine, cyanopyrazine, phenylacetonitrile, cyanopyrimidine, or 1H-imidazole-4-carbonitrile, wherein typical but non-limiting combinations comprise a combination of acetonitrile and cyanoquinoline, a combination of cyanopyridine and cyanopyrazine, or a combination of phenylacetonitrile and 1H-imidazole-4-carbonitrile.

In one embodiment, in step (a), a molar ratio of the diol to the nitrile compound is 1: (1-20), which may be, for example, 1:1, 1:2, 1:3, 1:4, 1:5, 1:6, 1:8, 1:10, 1:15, or 1:20, etc.: however, the molar ratio is not limited to the listed values, and other unlisted values within the value range are also applicable: optionally, the molar ratio is 1:5.

In one embodiment, in step (a), a feeding molar ratio of the diol to the CO₂ is 1: (1-10), which may be, for example, 1:1, 1:1.5, 1:2, 1:3, 1:4, 1:5, 1:6, 1:7, 1:8, 1:9, or 1:10, etc.; however, the feeding molar ratio is not limited to the listed values, and other unlisted values within the value range are also applicable: optionally, the feeding molar ratio is 1:1.5.

In one embodiment, in step (a), a temperature of the reaction unit is 50-200° C., which may be, for example, 50° C., 70° C., 90° C., 110° C., 130° C., 150° C., 170° C., or 200° C., etc.: however, the temperature is not limited to the listed values, and other unlisted values within the value range are also applicable: optionally, the temperature is 100-130° C.

A pressure is 100-1500 kPa, which may be, for example, 100 kPa, 200 kPa, 300 kPa, 400 kPa, 500 kPa, 800 kPa, 1000 kPa, or 1500 kPa, etc.: however, the pressure is not limited to the listed values, and other unlisted values within the value range are also applicable: optionally, the pressure is 500-800 kPa.

In one embodiment, in step (b), a pressure of the first separation device in the gas-liquid separation unit is 100-1500 kPa, which may be, for example, 100 kPa, 200 kPa, 300 kPa, 400 kPa, 500 kPa, 800 kPa, 1000 kPa, or 1500 kPa, etc.; however, the pressure is not limited to the listed values, and other unlisted values within the value range are also applicable.

In one embodiment, in step (b), a pressure of the second separation device is 100-600 kPa, which may be, for example, 100 kPa, 200 kPa, 300 kPa, 400 kPa, 500 kPa, or 600 kPa, etc.; however, the pressure is not limited to the listed values, and other unlisted values within the value range are also applicable: optionally, the pressure is 100-300 kPa.

In one embodiment, in step (c), an operating pressure of the light-component removal tower is 2-100 kPa, which may be, for example, 2 kPa, 4 kPa, 6 kPa, 8 kPa, 10 kPa, 20 kPa, 50 kPa, or 100 kPa, etc.; however, the operating pressure is not limited to the listed values, and other unlisted values within the value range are also applicable: optionally, the operating pressure is 2-5 kPa.

In one embodiment, in step (c), an operating pressure of the heavy-component removal tower is 2-100 kPa, which may be, for example, 2 kPa, 4 kPa, 6 kPa, 8 kPa, 10 kPa, 20 kPa, 50 kPa, or 100 kPa, etc.: however, the operating pressure is not limited to the listed values, and other unlisted values within the value range are also applicable: optionally, the operating pressure is 2-5 kPa.

In one embodiment, in step (c), an operating pressure of the high-purity tower is 2-100 kPa, which may be, for example, 2 kPa, 4 kPa, 6 kPa, 8 kPa, 10 kPa, 20 kPa, 50 kPa, or 100 kPa, etc.; however, the operating pressure is not limited to the listed values, and other unlisted values within the value range are also applicable: optionally, the operating pressure is 2-5 kPa.

In one embodiment, in step (d), the liquid phase extracted from the heavy-component removal tower is first fed into a crystallization device and then fed into a high-purity tower for separation and purification.

According to the requirements of product separation and purification, in order to avoid heavy-component byproducts or residual light-component solvents from being brought into the subsequent refining system and affecting the product quality, the refining unit may be provided with a crystallizer to realize the separation and purification of the product from the heavy components or light components. The crystallizer may be arranged between the heavy-component removal tower and the high-purity tower, which are connected by pipelines: a liquid inlet of the high-purity tower is connected by pipelines to at least one of a side liquid outlet or a bottom liquid outlet of the heavy-component removal tower, or an extraction outlet of the crystallizer. In particular, when a cyanopyridine solvent is used as an auxiliary for preparing cyclic ethylene carbonate, due to the high melting point of the by-product amide heavy component, the separation and purification of cyclic ethylene carbonate can be realized by a crystallizer, and then the purified cyclic ethylene carbonate can be refined by the high-purity tower to obtain a high-purity cyclic ethylene carbonate: however, if the crystallizer is not employed in the process, the purity of the finally obtained cyclic ethylene carbonate will not change much.

In the preparation process of cyclic propylene carbonate, because the cyanopyridine solvent and cyclic propylene carbonate are easy to form an azeotrope which is difficult to be separated by common distillation, the crystallizer and the high-purity tower need to be combined synergetically to achieve the preparation of high-purity cyclic propylene carbonate.

As an optional technical solution of the present application, the method comprises the following steps:
(a) introducing CO₂ gas from a gas inlet of the reaction unit, and feeding a mixed solution of a diol and a nitrile compound with a molar ratio of 1: (1-20) from a liquid inlet of the reaction unit, and performing diol esterifying cyclization with CO₂ to form cyclic carbonate under the action of a catalyst in the reaction unit: a feeding molar ratio of the diol to the CO₂ is 1: (1-10): the reaction unit has a temperature of 50-200° C. and a pressure of 100-1500 kPa:
(b) feeding a reaction material extracted from the reaction unit in step (a) into a first separation device with a pressure of 100-1500 kPa and a second separation device with a pressure of 100-600 kPa sequentially for gas-liquid separation, introducing a gas phase extracted from a gas outlet of the first separation device into the gas inlet of the reaction unit; and feeding a liquid phase extracted from a liquid outlet of the second separation device into a refining unit for refinement:
(c) subjecting a solution extracted from the liquid outlet of the second separation device in step (b) to a light-component removal tower with an operating pressure of 2-100 kPa, and extracting light components from a top liquid outlet of the light-component removal tower, and circulating to the reaction unit in step (a) for further reaction:
(d) feeding a cyclic carbonate solution extracted from a bottom liquid outlet of the light-component removal tower in step (c) into a heavy-component removal tower with an operating pressure of 2-100 kPa for removing heavy components, extracting the solution extracted from a top liquid outlet of the heavy-component removal tower or circulating to a bottom liquid inlet of the light-component removal tower for further separating light components out, and directly feeding a liquid phase extracted from the heavy-component removal tower into a high-purity tower with an operating pressure of 2-100 kPa for separation and purification, or first feeding into a crystallization device and then feeding into the high-purity tower with an operating pressure of 2-100 kPa for separation and purification and then extracting.

Compared to the related art, the present application at least has the following beneficial effects:
(1) in the device system for preparing cyclic carbonate by diol esterifying cyclization provided in the present application, CO₂ and the oversupply diol compound in the market are used as raw materials to produce cyclic carbonate, a high-value new energy solvent product, which is consistent with the concept of green, environmental-friendly, and low-carbon development, and expands the preparation route of cyclic carbonate:
(2) in the present application, the nitrile compound is used as an auxiliary, which can efficiently promote the diol esterifying cyclization with CO₂ to prepare cyclic carbonate, avoiding the use of the flammable and explosive epoxy alkanes as a reaction raw material, improving the safety of the cyclic carbonate production process, and facilitating the promotion and use in the market:
(3) in the present application, by using the gas-liquid separation unit and the refining unit, the separated CO₂ and nitrile compound are recycled, which substantially improves the conversion rate of raw materials, thus improving the productivity and economy of the process:
(4) the method for preparing cyclic carbonate by diol esterifying cyclization provided in the present application is an endothermic reaction, which has mild reaction conditions and simple separation processes, and the method is suitable for large-scale production.

Other aspects will be appreciated upon reading and understanding the accompanying drawings and detailed description.

### BRIEF DESCRIPTION OF DRAWINGS

The accompanying drawings are used to provide a further understanding of the technical solutions herein, form part of the specification, and explain the technical solutions herein in conjunction with the embodiments of the present application, and do not constitute a limitation of the technical solutions herein.

FIG. 1 is a schematic diagram of the device system for preparing cyclic carbonate by diol esterifying cyclization provided in Example 1 of the present application.

FIG. 2 is a schematic diagram of the device system for preparing cyclic carbonate by diol esterifying cyclization provided in Example 4 of the present application.

Reference list: 1-reaction unit: 2A-first separation tank: 2B—second separation tank: 3—light-component removal tower: 4—heavy-component removal tower: 5—high-purity tower: 6—compression unit: 7—first mechanical pump: 8—second mechanical pump; and 9—crystallization device.

### DETAILED DESCRIPTION

The technical solutions of the present application are further described below in conjunction with the accompanying drawings and via specific embodiments.

The technical solutions of the present application are further described below. However, the following examples are only simple examples of the present application, and do not represent or limit the protection scope of the present application, and the protection scope of the present application is defined by the claims.

It should be understood that in the description in the present application, the orientation or position relationship indicated by the terms, such as “center”, “longitudinal”, “lateral”, “up”, “down”, “front”, “back”, “left”, “right”, “vertical”, “horizontal”, “top”, “bottom”, “inside”, “outside”, etc., is based on the orientation or position relationship shown in the drawings, which is only intended for convenience and simplicity of the description, but not to indicate or imply that the device or element referred to must have a particular orientation or must be constructed and operated in a specific orientation, and therefore cannot be understood as a limitation of the present application. In addition, terms such as “first” and “second” are used only for descriptive purposes and cannot be understood as indicating or implying relative importance nor implicitly specifying the number of technical features referred to. Thus, features defined by “first” and “second” can explicitly or implicitly comprise one or more of the features. In the description of the present application, unless otherwise stated, “a plurality of” means two or more.

It should be noted that in the description in the present application, unless otherwise specified or limited, the terms such as “arrange”, “connect” and “attach” are to be understood in a broad sense, for example, as a fixed connection, or as a detachable connection, or as an integrated connection; as a mechanical connection, or as an electrical connection: as a direct connection, or as an indirect connection via an intermediate medium, or as a communication between two elements. For those skilled in the field, the specific meaning of the above terms can be understood in the light of specific cases in the present application.

It should be understood by those skilled in the field that the present application includes the necessary pipelines, regular valves and general pumps for the process integrity, but the above is not the core idea of the present application. Those skilled in the field can make their own arrangement based on the process and equipment: the present application does not make any special requirements and specific limitations thereon.

### Example 1

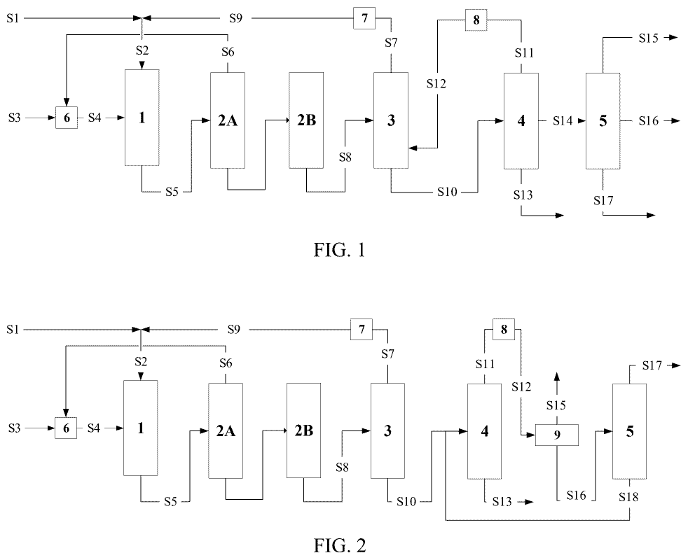

This example provides a device system for preparing cyclic carbonate by diol esterifying cyclization, the schematic diagram of which is shown in FIG. 1.

The device system comprises a reaction unit 1, a gas-liquid separation unit, and a refining unit which are connected in sequence:

the gas-liquid separation unit comprises a first separation tank 2A and a second separation tank 2B which are arranged in series:

the refining unit comprises a light-component removal tower 3, a heavy-component removal tower 4, and a high-purity tower 5 which are connected in sequence.

the reaction unit 1 is a tubular fixed-bed reactor, which has an immobilized heterogeneous catalyst inside and maintains the reaction temperature by 150° C. steam;

a gas outlet of the first separation device 2A is connected to a gas inlet of the reaction unit 1 via a compression device 6:

a liquid outlet of the light-component removal tower 3 is connected to a liquid inlet of the reaction unit 1;

the heavy-component removal tower 4 is connected to a bottom part of the light-component removal tower 3.

In this example, the loading of immobilized heterogeneous catalyst is realized by adopting a tubular fixed-bed reactor, avoiding the problem of catalyst separation, and simplifying the process: meanwhile, the heat medium uniformly supplies heat to the reaction process via the shell cavity of the shell-and-tube reactor, the heat transfer efficiency is high, and the temperature of the reaction process can be controlled stably.

### Example 2

This example provides a device system for preparing cyclic carbonate by diol esterifying cyclization. The device system is the same as that of Example 1 except that the reaction unit 1 is a bubbling-bed reactor, the diol esterifying cyclization is catalyzed by continuous supply of a homogeneous catalyst, and the bed temperature of the reactor is maintained by using 150° C. heat transfer oil in the jacket or built-in heat exchanger of the bubbling-bed reactor.

In this example, the CO₂ gas fully contacts with the diol raw material and the homogeneous catalyst by using the bubbling-bed reactor, and the high gas-liquid mass transfer rate promotes the increase of reaction rate; meanwhile, the heat medium provides heat to the reaction by the jacket shell or built-in heat exchanger of the bubbling-bed reactor, which is simple in structure, low in cost, and suitable for large scale production.

### Example 3

This example provides a device system for preparing cyclic carbonate by diol esterifying cyclization. The device system is the same as that of Example 1 except that the reaction unit 1 is the tubular fixed-bed reactor, tubes are provided with inert random metal packings inside and a homogeneous catalyst is consistently supplied to catalyze the diol esterifying cyclization, and the bed temperature of the reactor is maintained by using 150° C. medium-pressure boiler hot water in the shell layer.

In this example, the tubular fixed-bed reactor and the system thereof are used, which uses the metal filler packed in the tubes of the reactor to enhance the gas liquid contact mass transfer efficiency, and has the advantages of high activity and uniform contact of the homogeneous catalyst, so as to ensure a uniform reaction rate in the reactor.

### Example 4

This example provides a device system for preparing cyclic carbonate by diol esterifying cyclization, the schematic diagram of which is shown in FIG. 2.

The device system is the same as that of Example 1 except that the reaction unit 1 is a fluidized-bed reactor, which is filled with a granular or powdery heterogeneous catalyst to catalyze the diol esterifying cyclization, and the bed temperature of the reactor is maintained by using 150° C. steam; and

the refining unit further comprises a crystallization device 9: the crystallization device 9 is arranged between a heavy-component removal tower 4 and a high-purity tower 5: the crystallization device 9 is an evaporation crystallizer; and the high-purity tower 5 is connected to the heavy-component removal tower 4.

In this example, the type of the reaction unit is a fluidized-bed reactor, which is mainly suitable for a heterogeneous catalyst system with a small particle size, the contact area among the gas liquid solid phases is large, and the internal diffusion resistance of reaction mass transfer is small, so that the reaction mass transfer and heat transfer rates can be greatly improved, and the efficiency of the catalyst can be fully utilized. During the reaction process, the reactor is preloaded with heterogeneous catalyst particles, and the catalyst particles are driven by the gas and liquid flow and flow in the bed, which realizes the improvement of reaction mass transfer and heat transfer efficiency.

### Example 5

This example provides a method for preparing cyclic carbonate by diol esterifying cyclization, and the method is performed with the device system for preparing cyclic carbonate by diol esterifying cyclization in Example 1, which specifically comprises the following steps:
(1) cyanopyridine was used as an auxiliary, the reaction raw material of ethylene glycol s1 had a feeding flow rate of 96 kg/h and CO₂ (s3) had a feeding flow rate of 66.3 kg/h, tubes of the tubular fixed-bed reactor were filled with a heterogeneous catalyst ZnO/Al2O3, an operating pressure was 500 kPa, and the reaction temperature was controlled at 100° C. with medium-pressure steam, and at the reactor inlet, a molar ratio of ethylene glycol to cyanopyridine was controlled to be 1:5 and a molar ratio of ethylene glycol to CO₂ was controlled to be 1:1.5: the ethylene carbonate solution s5 containing 6.9% CO₂ extracted from the reactor outlet was fed into a first separation tank 2A for gas-liquid separation, an operating pressure of the first separation tank 2A was controlled to be 500 kPa for adiabatic flash evaporation, and 96% of the CO₂ (s6) was extracted from the top of the first separation tank 2A and circulated back to the reactor for further use by a compressor 6, and a total gas feed into the reactor was s4: a pressure of a second separation tank 2B was controlled to be 300 kPa, and a liquid phase extracted from a bottom liquid outlet of the first separation tank 2A was fed into the second separation tank 2B to continue to separate a residual gas component:
(2) the liquid phase s8 extracted from the second separation tank 2B in step (1) was fed into a light-component removal tower 3 for separation, wherein the feed material s8 contained 7.4% of ethylene carbonate and 77.4% of cyanopyridine, the light-component removal tower 3 was controlled to have a top operating pressure of 4 kPa and a top temperature of 118° C., the number of theoretical plates was 21, and the feed position was the ninth plate, the gas phase extracted from the tower top was condensed and 70% of the material s7 was pressurized by a first mechanical pump 7 (s9) and circulated back to the reactor, a total liquid feed into the reactor was s2, and 30% of the material was circulated back to the top of the light-component removal tower 3 by a pressure reducing valve:
(3) the ethylene carbonate solution s10 extracted from the bottom tower kettle of light-component removal tower 3 was fed into the heavy-component removal tower 4, the top operating pressure was controlled to be 2 kPa, the number of theoretical plates was 18, and the feed position was the 13th plate, 99% of ethylene carbonate solution s11 extracted from the top of the heavy-component removal tower was pressurized by a mechanical pump 8 (s12) and circulated back to the light-component removal tower, 97.2% of an amide heavy component s13 was extracted from the bottom tower kettle and fed to an auxiliary regeneration unit, and 99.9% of ethylene carbonate was extracted from the side:
(4) the ethylene carbonate s14 extracted from the side of the heavy-component removal tower 4 was fed into the high-purity tower 5, the top operating pressure was controlled to be 3 kPa, the number of theoretical plates was 25, and the feed position was the 20th plate, and the top material s15 and the bottom material s17 were extraction outlets for the light component and the heavy component, respectively, and 131.4 kg/h of the product ethylene carbonate s16 was extracted from the side.

In this example, a yield of ethylene carbonate was 96.5%, and a purity of the extracted ethylene carbonate product was greater than 99.99 wt %. The key materials and component calculation results of this example are shown in Table 1.

TABLE 1

| Material name | Unit | s1 | s2 | s3 | s4 | s5 | s6 | s7 | s8 | s9 |
|---|---|---|---|---|---|---|---|---|---|---|
| Temperature | ° C. | 46.2 | 92.4 | 45 | 169.3 | 100 | 100 | 100 | 100 | 101 |
| Pressure | kPa | 800 | 800 | 1301.3 | 1601.3 | 500 | 480 | 3 | 480 | 800 |
| Mass | kg/hr | 96 | 1856.7 | 66.3 | 214.3 | 2071.0 | 149.1 | 1602.2 | 1921.9 | 1602.2 |
| flow rate | | | | | | | | | | |
| Mass | CO₂ | 0 | 0 | 99.94 | 97.55 | 6.90 | 95.77 | 0 | 0.01 | 0 |
| fraction | Ethylene | 100 | 10.59 | 0 | 0 | 4.86 | 0.04 | 6.28 | 5.23 | 6.28 |
| % | glycol | | | | | | | | | |
| | Cyano- | 0 | 88.66 | 0 | 0.03 | 71.90 | 0.71 | 92.85 | 77.42 | 92.85 |
| | pyridine | | | | | | | | | |
| | Ethylene | 0 | 0.53 | 0 | 0 | 6.87 | 0.01 | 0.62 | 7.41 | 0.62 |
| | carbonate | | | | | | | | | |

| Material name | Unit | s10 | s11 | s12 | s13 | s14 | s15 | s16 | s17 |
|---|---|---|---|---|---|---|---|---|---|
| Temperature | ° C. | 168.4 | 125.3 | 125.3 | 184.3 | 129.7 | 133.8 | 139.9 | 143.3 |
| Pressure | kPa | 6 | 2 | 10 | 3 | 2.4 | 3 | 3.7 | 4 |
| Mass | kg/hr | 328.2 | 9.3 | 9.3 | 186.5 | 132.4 | 0.8 | 131.4 | 0.2 |
| flow rate | | | | | | | | | |
| Mass | CO₂ | 0 | 0 | 0 | 0 | 0 | 0 | 0 | 0 |
| fraction | Ethylene | 0 | 0 | 0 | 0 | 0 | 0 | 0 | 0 |
| % | glycol | | | | | | | | |
| | Cyano- | 0.03 | 0.53 | 0.53 | 0 | 0.04 | 5.33 | 0.01 | 0 |
| | pyridine | | | | | | | | |
| | Ethylene | 43.17 | 99.00 | 99.00 | 0.08 | 99.93 | 91.38 | 99.99 | 90.75 |
| | carbonate | | | | | | | | |

### Example 6

This example provides a method for preparing cyclic carbonate by diol esterifying cyclization, and the method is performed with the device system for preparing cyclic carbonate by diol esterifying cyclization in Example 2, which specifically comprises the following steps:
(1) cyanopyrazine was used as an auxiliary, the reaction raw material of ethylene glycol s1 had a feeding flow rate of 96 kg/h and CO₂ (s3) had a feeding flow rate of 65.9 kg/h, an operating pressure of the bubbling-bed reactor was 800 kPa, and the reaction temperature was controlled at 130° C. by using heat transfer oil through the heating jacket, and in the bubbling-bed reactor, a molar ratio of ethylene glycol to cyanopyrazine was controlled to be 1:5 and a molar ratio of ethylene glycol to CO₂ was controlled to be 1:1.5: CO₂ and ethylene glycol and CO₂ were continuously conversed to prepare ethylene carbonate by the continuous supplementation of the homogeneous composite catalyst, tin bromide/trialkylethyl phosphonium bromide: the ethylene carbonate solution s5 containing 6.3% CO₂ extracted from the reactor outlet was fed into a first separation tank 2A for gas-liquid separation, an operating pressure of the first separation tank 2A was controlled to be 800 kPa, and 96% of the CO₂ (s6) extracted from the top of the first separation tank was circulated back to the reactor for further reaction through a compressor 6, and a total gas feed into the reactor was s4: a pressure of the second separation tank 2B was controlled to be 100 kPa, and a liquid phase extracted from a bottom liquid outlet of the first separation tank 2A was fed into the second separation tank 2B to continue to separate a residual gas component:
(2) the liquid phase extracted from the second separation tank 2B in step (1) was fed into a light-component removal tower 3 for separation, wherein the feed material s8 contained 5.8% of ethylene carbonate and 78.5% of cyanopyridine, a top operating pressure was controlled to be 4 kPa, a top temperature was controlled to be 118° C., the number of theoretical plates was 21, and the feed position was the 9th plate, the gas phase extracted from the tower top was condensed and 70% of the material s7 was pressurized by a first mechanical pump 7 (s9) and circulated back to the reactor, a total liquid feed into the reactor was s2, and 30% of the material was circulated back to the top of the light-component removal tower 3 by a pressure reducing valve:
(3) the ethylene carbonate solution s10 extracted from the bottom tower kettle of the light-component removal tower 3 was fed into the heavy-component removal tower 4, the top operating pressure was controlled to be 2 kPa, the number of theoretical plates was 18, and the feed position was the 13th plate, 99% of ethylene carbonate solution s11 extracted from the top of the heavy component removal tower was pressurized by a mechanical pump 8 (s12) and circulated back to the light component removal tower, 96.3% of the heavy component s13 was extracted from the bottom tower kettle of the heavy-component removal tower 4 and fed to an auxiliary regeneration unit, and the ethylene carbonate solution s14 extracted from the side had a purity of 99.9%:
(4) the ethylene carbonate material s14 extracted from the side of the heavy-component removal tower 4 was fed into the high-purity tower 5 for further refining, the top operating pressure of the high-purity tower 5 was controlled to be 3 kPa, the number of theoretical plates was 25, and the feed position was the 20th plate, and the top material s15 and the bottom material s17 were extraction outlets for the light component and the heavy component, respectively, and 129.5 kg/h of the product ethylene carbonate s16 was extracted from the side of the high-purity tower 5.

In this example, a yield of ethylene carbonate was 95%, and a purity of the extracted ethylene carbonate product was 99.99 wt %. The key material and component calculation results of this example are shown in Table 2.

TABLE 2

| Material name | Unit | s1 | s2 | s3 | s4 | s5 | s6 | s7 | s8 | s9 |
|---|---|---|---|---|---|---|---|---|---|---|
| Temperature | ° C. | 46.2 | 93.5 | 45 | 169.7 | 100 | 100 | 100 | 100 | 100 |
| Pressure | kPa | 800 | 800 | 1301.3 | 1601.3 | 500 | 480 | 3 | 480 | 800 |
| Mass | kg/hr | 96 | 2092.4 | 65.9 | 239 | 2331.4 | 174.4 | 1838.5 | 2157 | 1838.5 |
| flow rate | | | | | | | | | | |
| Mass | CO₂ | 0 | 0 | 99.94 | 97.60 | 7.19 | 96.00 | 0 | 0.01 | 0 |
| fraction | Ethylene | 100 | 10.55 | 0 | 0.01 | 5.36 | 0.04 | 6.79 | 5.79 | 6.79 |
| % | glycol | | | | | | | | | |
| | Cyano- | 0 | 88.51 | 0 | 0.03 | 72.73 | 0.72 | 92.15 | 78.56 | 92.15 |
| | pyrazine | | | | | | | | | |
| | Ethylene | 0 | 0.67 | 0 | 0 | 6.25 | 0.01 | 0.76 | 6.75 | 0.76 |
| | carbonate | | | | | | | | | |

| Material name | Unit | s10 | s11 | s12 | s13 | s14 | s15 | s16 | s17 |
|---|---|---|---|---|---|---|---|---|---|
| Temperature | ° C. | 168.5 | 125.4 | 125.4 | 183.8 | 129.7 | 133.8 | 139.9 | 141.9 |
| Pressure | kPa | 6 | 2 | 10 | 3 | 2.4 | 3 | 3.7 | 4 |
| Mass | kg/hr | 326.5 | 9.1 | 9.1 | 186.5 | 130.9 | 0.8 | 129.5 | 0.64 |
| flow rate | | | | | | | | | |
| Mass | CO₂ | 0 | 0 | 0 | 0 | 0 | 0 | 0 | 0 |
| fraction | Ethylene | 0 | 0 | 0 | 0 | 0 | 0 | 0 | 0 |
| % | glycol | | | | | | | | |
| | Cyano- | 0.03 | 0.55 | 0.55 | 0 | 0.04 | 5.35 | 0.01 | 0 |
| | pyrazine | | | | | | | | |
| | Ethylene | 43.05 | 99.08 | 99.08 | 0.37 | 99.94 | 92.06 | 99.99 | 98.67 |
| | carbonate | | | | | | | | |

### Example 7

This example provides a method for preparing cyclic carbonate by diol esterifying cyclization, and the method is performed with the device system for preparing cyclic carbonate by diol esterifying cyclization in Example 3, which specifically comprises the following steps:
(1) cyanopyrimidine was used as a reaction auxiliary, the reaction raw material of ethylene glycol s1 had a feeding flow rate of 144 kg/h and CO₂ (s3) had a feeding flow rate of 97.2 kg/h, tubes of the tubular fixed-bed reactor were filled with an inert metal filler to enhance gas-liquid distribution and heat transfer, wherein an operating pressure of the reactor was controlled to be 1500 kPa, and the reaction temperature was controlled at 100° C. by using the medium-pressure hot water in the reactor shell layer, and a molar ratio of cyanopyrimidine to ethylene glycol was controlled to be 5:1 and a molar ratio of CO₂ to ethylene glycol was controlled to be 1.5:1: CO₂ and ethylene glycol were continuously conversed to prepare ethylene carbonate by the continuous supplementation of the homogeneous composite catalyst, zinc bromide/tetrabutylphosphonium bromide: the ethylene carbonate solution s5 containing 7.4% CO₂ extracted from the reactor outlet was fed into a first separation tank 2A for gas-liquid separation, an operating pressure of the first separation tank 2A was controlled to be 1500 kPa, and 96% of the CO₂ (s6) extracted from the top of the first separation tank was circulated back to the reactor for further reaction through a compressor 6, and a total gas feed into the reactor was s4: a pressure of the second separation tank 2B was controlled to be 200 kPa, and a liquid phase extracted from a bottom liquid outlet of the first separation tank 2A was fed into the second separation tank 2B to continue to separate a residual gas component:
(2) the liquid phase extracted from the second separation tank 2B in step (1) was fed into a light-component removal tower 3 for separation, wherein the feed material s8 contained 6.6% of ethylene carbonate and 78.8% of cyanopyrimidine, a top operating pressure of the light-component removal tower 3 was controlled to be 8 kPa, the number of theoretical plates was 21, and the feed position was the 9th plate, the gas phase extracted from the tower top was condensed, and 50% of the material s7 was pressurized by a first mechanical pump 7 (s9) and circulated back to the reactor, a total liquid feed into the reactor was s2, and 50% of the material was circulated back to the top of the light-component removal tower 3 through a pressure reducing valve:
(3) the ethylene carbonate solution s10 extracted from the bottom tower kettle of the light-component removal tower 3 was fed into the heavy-component removal tower 4, the top operating pressure of the heavy-component removal tower 4 was controlled to be 2 kPa, the number of theoretical plates was 18, and the feed position was the 13th plate, 99.5% of the ethylene carbonate solution s11 extracted from the top of the heavy-component removal tower was pressurized by a mechanical pump 8 (s12) and circulated back to the light-component removal tower, 95.5% of a heavy component s13 extracted from the bottom tower kettle of the heavy-component removal tower 4 was fed to an auxiliary regeneration unit, and 99.5% of ethylene carbonate s14 was extracted from the side liquid outlet;
(4) the ethylene carbonate solution s14 extracted from the side liquid outlet of the heavy-component removal tower 4 was fed into the high-purity tower 5 for further refining and separation, the top operating pressure of the high purity tower 5 was controlled to be 3 kPa, the number of theoretical plates was 25, and the feed position was the 20th plate, and the top material s15 and the bottom material s17 were extraction outlets of the light component and the heavy component, respectively, and 192 kg/h of the ethylene carbonate product s16 was extracted from the side of the high purity tower 5.

In this example, a yield of ethylene carbonate was 94%, and a purity of the extracted ethylene carbonate product was 99.99 wt %. The key material and component calculation results of this example are shown in Table 3.

TABLE 3

| Material name | Unit | s1 | s2 | s3 | s4 | s5 | s6 | s7 | s8 | s9 |
|---|---|---|---|---|---|---|---|---|---|---|
| Temperature | ° C. | 46.2 | 93.4 | 45 | 169.7 | 100 | 100 | 100 | 100 | 100 |
| Pressure | kPa | 800 | 800 | 1301.3 | 1601.3 | 500 | 480 | 3 | 480 | 800 |
| Mass | kg/hr | 144 | 3252.1 | 97.2 | 372.3 | 3624.4 | 277.1 | 2873.2 | 3347.3 | 2873.2 |
| flow rate | | | | | | | | | | |
| Mass | CO₂ | 0 | 0 | 99.94 | 97.71 | 7.36 | 96.20 | 0 | 0.01 | 0 |
| fraction | Ethylene | 100 | 10.51 | 0 | 0 | 5.47 | 0.04 | 6.89 | 5.92 | 6.89 |
| % | glycol | | | | | | | | | |
| | Cyano- | 0 | 88.31 | 0 | 0.03 | 72.82 | 0.72 | 91.78 | 78.79 | 91.78 |
| | pyrimidine | | | | | | | | | |
| | Ethylene | 0 | 0.85 | 0 | 0 | 6.11 | 0.01 | 0.96 | 6.62 | 0.96 |
| | carbonate | | | | | | | | | |

| Material name | Unit | s10 | s11 | s12 | s13 | s14 | s15 | s16 | s17 |
|---|---|---|---|---|---|---|---|---|---|
| Temperature | ° C. | 183.4 | 125.4 | 125.4 | 183.9 | 129.8 | 133.8 | 139.9 | 172.4 |
| Pressure | kPa | 10 | 2 | 12 | 3 | 2.4 | 3 | 3.7 | 4 |
| Mass | kg/hr | 482.3 | 9.9 | 9.9 | 278.5 | 193.9 | 0.8 | 192 | 1.1 |
| flow rate | | | | | | | | | |
| Mass | CO₂ | 0 | 0 | 0 | 0 | 0 | 0 | 0 | 0 |
| fraction | Ethylene | 0 | 0 | 0 | 0 | 0 | 0 | 0 | 0 |
| % | glycol | | | | | | | | |
| | Cyano- | 0.02 | 0.50 | 0.50 | 0 | 0.04 | 7.03 | 0.01 | 0 |
| | pyrimidine | | | | | | | | |
| | Ethylene | 42.28 | 99.48 | 99.48 | 0.41 | 99.48 | 92.78 | 99.99 | 15.75 |
| | carbonate | | | | | | | | |

### Example 8

This example provides a method for preparing cyclic carbonate by diol esterifying cyclization, and the method is performed with the device system for preparing cyclic carbonate by diol esterifying cyclization in Example 4, which specifically comprises the following steps:
(1) cyanopyridine was used as a reaction auxiliary, and the reaction raw material 1.2-propylene glycol s1 had a feeding flow rate of 96 kg/h, the fluidized-bed reactor was filled with heterogeneous catalyst ZnO particles, the reactor pressure was controlled to be 1500 kPa by the CO₂ (s3) inlet-gas flow: the reaction temperature was maintained at 180° C. by introducing low-pressure steam into the jacket, and by controlling the feeding flow: a molar ratio of cyanopyrimidine to 1.2-propylene glycol in the reactor was 5:1 and a molar ratio of CO₂ to 1.2-propylene glycol in the reactor was 1.5:1: the propylene carbonate solution s5 containing 18.1% CO₂ extracted from the reactor outlet was fed into a first separation tank 2A for gas-liquid separation, an operating pressure of the first separation tank 2B was controlled to be 800 kPa. and 80.6% of the CO₂ (6) extracted from the top of the first separation tank 2A was circulated back to the reactor for further reaction through a compressor 6, and a total gas feed into the reactor was s4: a pressure of the second separation tank 2B was controlled to be 100 kPa. and a liquid phase extracted from a bottom liquid outlet at the of the first separation tank 2A was fed into the second separation tank 2B to continue to separate a residual gas component:
(2) the liquid phase extracted from the second separation tank 2B in step (1) was fed into a light-component removal tower 3 for separation, wherein the feed material s8 contained 14.5% of propylene carbonate and 59.8% of cyanopyridine, the light-component removal tower 3 was controlled to have a top operating pressure of 4 kPa and a top temperature of 123° C. the number of theoretical plates was 60, and the feed position was the 6th plate, the gas phase extracted from the tower top was condensed and 575 kg/h of the cyanopyridine material s7 was pressurized by a first mechanical pump 7 (s9) and circulated back to the reactor, a total liquid feed into the reactor was s2: 303 kg/h of a mixed solution s10 of the propylene carbonate, the by-product amide heavy component, and a little residual cyanopyridine extracted from the bottom liquid outlet of the light-component removal tower 3 was fed to the heavy-component removal tower 4 for further separation:
(3) the propylene carbonate solution s10 extracted from the bottom tower kettle of the light-component removal tower 3 was fed into the heavy-component removal tower 4, the top operating pressure of the heavy-component removal tower 4 was controlled to be 2 kPa, the number of theoretical plates was 20, and the feed position was the 15th plate. 137 kg/h of the amide heavy component s13 extracted from the bottom tower kettle of the heavy-component removal tower 4 was fed to an auxiliary regeneration unit, 159.1 kg/h of 86% propylene carbonate s12 extracted from the top of the heavy component removal tower 4 was fed into the crystallizer 9 and the high-purity tower 5 successively for further separation and refining:
(4) the material s12 extracted from the tower top of the heavy-component removal tower 4 was subjected to cooling crystallization in the crystallizer 9 to separate the residual cyanopyridine from the product propylene carbonate, wherein the mother liquor s15 (23 kg/h) in the crystallizer 9 was extracted from the mother liquor extraction outlet and fed to the light-component removal tower 3 to continue the separation and refining, and the crystal liquid s16 (136 kg/h) was fed to the high-purity tower 5 to continue the purification and refining through the crystal liquid extraction outlet s16: the top operating pressure of the high-purity tower 5 was controlled to be 2 kPa, the number of theoretical plates was 25, and the feed position was the 3rd plate: 126 kg/h of the propylene carbonate product solution s17 was extracted from the tower top, the solution containing a little heavy component s18 extracted from the bottom tower kettle was pressurized and circulated back to the heavy-component removal tower 4 to continue the separation.

In this example, a yield of ethylene carbonate was 97.9%, and a purity of the extracted ethylene carbonate product was 99.94 wt %. The key material and component calculation results of this example are shown in Table 4.

TABLE 4

| Material name | Unit | s1 | s2 | s3 | s4 | s5 | s6 | s7 | s8 |
|---|---|---|---|---|---|---|---|---|---|
| Temperature | ° C. | 39.6 | 100 | 45 | 174.3 | 100 | 45 | 30.9 | 120 |
| Pressure | kPa | 800 | 780 | 1301.3 | 1601.3 | 500 | 470 | 4 | 8 |
| Mass | kg/hr | 248.6 | 823.6 | 55.5 | 165.7 | 989.3 | 110.9 | 575 | 877.9 |
| flow rate | | | | | | | | | |
| Mass | CO₂ | 0 | 0.02 | 99.98 | 87.42 | 9.10 | 80.62 | 0.02 | 0.01 |
| fraction | 1,2-Propylene | 61.39 | 79.73 | 0 | 0.02 | 53.15 | 0.51 | 87.65 | 59.83 |
| % | glycol | | | | | | | | |
| | Cyano- | 0 | 0.01 | 0 | | 12.89 | 0.04 | 0.01 | 14.52 |
| | pyridine | | | | | | | | |
| | Propylene | 38.60 | 20.24 | 0 | 0 | 7.16 | 0.09 | 12.30 | 8.06 |
| | carbonate | | | | | | | | |

| Material name | Unit | s9 | s10 | s12 | s13 | s15 | s16 | s17 | s18 |
|---|---|---|---|---|---|---|---|---|---|
| Temperature | ° C. | 32.5 | 163.3 | 118.5 | 174.5 | 119.6 | 119.6 | 118.4 | 135 |
| Pressure | kPa | 800 | 600 | 2 | 2 | 800 | 800 | 2 | 4 |
| Mass | kg/hr | 575 | 302.9 | 159.1 | 153.8 | 22.9 | 136.2 | 126.2 | 10 |
| flow rate | | | | | | | | | |
| Mass | CO₂ | 0.02 | 0 | 0 | 0 | 0 | 0 | 0 | 0 |
| fraction | 1,2-Propylene | 87.65 | 7.02 | 13.45 | 0 | 92.45 | 0.16 | 0.06 | 1.40 |
| % | glycol | | | | | | | | |
| | Cyano- | 0.01 | 42.08 | 86.33 | 0 | 5.99 | 99.84 | 99.94 | 98.60 |
| | pyridine | | | | | | | | |
| | Propylene | 12.30 | 0 | 0 | 0 | 0 | 0 | 0 | 0 |
| | carbonate | | | | | | | | |

To sum up, for the device system for preparing cyclic carbonate by diol esterifying cyclization of the present application, the CO₂ and diol compound are used as raw materials, and the nitrile compound is used as an auxiliary to produce cyclic carbonate, a high-value new energy solvent product, which is consistent with the concept of green, environmental-friendly, and low-carbon development, and also avoids the use of flammable and explosive epoxy alkanes as a reaction raw material, and improves the safety of the cyclic carbonate production process: moreover, by using the gas-liquid separation unit and the refining unit, the separated CO₂ and nitrile compounds are recycled, which substantially improves the raw material conversion rate, improves the productivity and economy of the process, and has a prospect of large-scale popularization and application.

The applicant declares that detailed structural features of the present application are illustrated by the above examples in the present application, but the present application is not limited to the above detailed structural features, that is, the present application does not necessarily rely on the above detailed structural features to be implemented. Those skilled in the art should understand that any improvement of the present application, the equivalent substitution of selected parts, the addition of auxiliary parts, and the selection of specific methods in the present application shall fall within the protection scope and disclosure scope of the present application.

The above describes the preferred embodiments of the present application in detail. However, the present application is not limited to the specific details in the above embodiments, and within the scope of the technical conception of the present application, a plurality of simple variations of the technical solution of the present application may be performed, and all simple variations fall within the protection scope of the present application.

It should also be noted that each of specific technical features described in the above specific embodiments can be combined in any suitable way without contradiction, and in order to avoid unnecessary repetition, the present application will not separately describe the various possible combinations.

In addition, combinations among the various different embodiments of the present application can also be made without limitation, and as long as they do not contradict the idea of the present application, they should be regarded as the disclosure in the present application.

1. A device system for preparing cyclic carbonate by diol esterifying cyclization, which comprises a reaction unit, a gas-liquid separation unit, and a refining unit which are connected in sequence;
in the reaction unit, a nitrile compound is used as an auxiliary to synthesize cyclic carbonate by diol esterifying cyclization with CO₂; the reaction unit comprises any one of a fixed-bed reactor, a bubbling-bed reactor, or a fluidized-bed reactor; and a feeding/discharging method for liquid of the reaction unit comprises top liquid-feeding and bottom liquid-discharging, or bottom liquid-feeding and top liquid-discharging; the gas-liquid separation unit comprises a first separation device and a second separation device which are arranged in series;
the refining unit comprises a light-component removal tower, a heavy-component removal tower, and a high-purity tower which are arranged in sequence;
a gas outlet of the first separation device is connected to a gas inlet of the reaction unit via a compression device; a liquid outlet of the light-component removal tower is connected to a liquid inlet of the reaction unit; the heavy-component removal tower is connected to a bottom part of the light-component removal tower.

2. The device system according to claim 1, wherein the reaction unit comprises any one of a jacketed heat exchanger, a shell-and-tube heat exchanger, or a built-in heat exchanger;
in a case where the reaction unit is the fixed-bed reactor, the feeding/discharging method is top liquid-feeding and bottom liquid-discharging:
in a case where the reaction unit is the bubbling-bed reactor, the feeding/discharging method is bottom liquid-feeding and top liquid-discharging;
in a case where the reaction unit is the fluidized-bed reactor, the feeding/discharging method is bottom liquid-feeding and top liquid-discharging.

3. The device system according to claim 1, wherein the refining unit further comprises a crystallization device;
the crystallization device comprises an evaporation crystallizer or a cooling crystallizer.

4. The device system according to claim 3, wherein the crystallization device is arranged between the heavy-component removal tower and the high-purity tower.

5. The device system according to claim 1, wherein the high-purity tower is connected to the heavy-component removal tower.

6. (canceled)

7. (canceled)

8. (canceled)

9. (canceled)

10. (canceled)

11. (canceled)

12. A method for preparing cyclic carbonate by diol esterifying cyclization, which is performed with the device system for preparing cyclic carbonate by diol esterifying cyclization according to claim 1;
the method comprises using a nitrile compound as an auxiliary to realize diol esterifying cyclization for synthesizing cyclic carbonate, and the specific reaction is as follows: 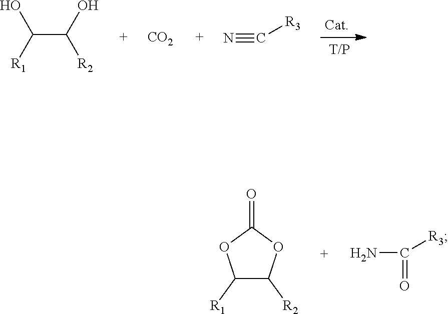
wherein the diol comprises a vicinal diol;
in the molecular structure of the diol, R₁ and R₂ comprise any one of hydrogen, methyl, ethyl, or propyl;
in the molecular structure of the nitrile compound, R₃ comprises any one of ethyl, phenylmethyl, pyridine, pyrimidine, pyrazine, imidazole, or quinoline.

13. The method according to claim 12, wherein the method comprises the following steps:
(a) introducing CO₂ gas from a gas inlet of a reaction unit, and feeding a mixed solution of a diol and a nitrile compound from a liquid inlet of the reaction unit, and performing diol esterifying cyclization with CO₂ to form cyclic carbonate under the action of a catalyst in the reaction unit;
wherein the reaction unit is filled with a heterogeneous catalyst and/or a homogeneous catalyst;
(b) feeding a reaction material extracted from the reaction unit in step (a) into a first separation device and a second separation device sequentially for gas-liquid separation, introducing a gas phase extracted from a gas outlet of the first separation device into a gas inlet of the reaction unit; and feeding a liquid phase extracted from a liquid outlet of the second separation device into a refining unit for refinement;
(c) subjecting a solution extracted from the liquid outlet of the second separation device in step (b) to a light-component removal tower, and extracting light components from a top liquid outlet of the light-component removal tower and circulating to the reaction unit in step (a) for further reaction; and
(d) feeding a cyclic carbonate solution extracted from a bottom liquid outlet of the light-component removal tower in step (c) into the heavy-component removal tower for removing heavy components, extracting a solution from a top liquid outlet of the heavy-component removal tower or circulating to a bottom liquid inlet of the light-component removal tower for further separating light components out, and feeding a liquid phase extracted from the heavy-component removal tower into a high-purity tower for separation and purification and then extracting.

14. The method according to claim 13, wherein
the heterogeneous catalyst comprises any one or a combination of at least two of silicon oxide, aluminum oxide, iron oxide, copper oxide, zinc oxide, tin oxide, lanthanum oxide, cerium oxide, cobalt oxide, dialkylzinc oxide, dialkyltin oxide, dialkyllanthanum oxide, dialkylcerium oxide, or dialkylcobalt oxide.

15. The method according to claim 13, wherein in step (a), a molar ratio of the diol to the nitrile compound is 1: (1-20).

16. The method according to claim 13, wherein a shape of the heterogeneous catalyst comprises any one or a combination of at least two of powder, granule, sphere, rod, cube, or polyhedron.

17. The method according to claim 13, wherein the homogeneous catalyst comprises any one or a combination of at least two of zinc bromide, stannic bromide, cerium bromide, tetraalkyl phosphonium bromide, trialkylethyl phosphonium bromide, tetraphenyl phosphonium bromide, triphenylbutyl phosphonium bromide, zinc acetate, tin acetate, or cerium acetate.

18. The method according to claim 13, wherein the nitrile compound comprises any one or a combination of at least two of acetonitrile, cyanoquinoline, cyanopyridine, cyanopyrazine, phenylacetonitrile, cyanopyrimidine, or 1H-imidazole-4-carbonitrile.

19. The method according to claim 13, wherein in step (a), a feeding molar ratio of the diol to the CO₂ is 1:(1-10).

20. The method according to claim 13, wherein in step (a), the reaction unit has a temperature of 50-200° C. and a pressure of 100-1500 kPa.

21. The method according to claim 13, wherein in step (b), a pressure of the first separation device in the gas-liquid separation unit is 100-1500 kPa.

22. The method according to claim 13, wherein in step (b), a pressure of the second separation device is 100-600 kPa.

23. The method according to claim 13, wherein in step (c), an operating pressure of the light-component removal tower is 2-100 kPa.

24. The method according to claim 13, wherein in step (c), an operating pressure of the heavy-component removal tower is 2-100 kPa.

25. The method according to claim 13, wherein in step (c), an operating pressure of the high-purity tower is 2-100 kPa.

26. The method according to claim 13, wherein in step (d), the liquid phase extracted from the heavy-component removal tower is first fed into a crystallization device and then fed into the high-purity tower for separation and purification.
